(19) United States
(12) Patent Application Publication
(10) Pub. No.: US 2007/0225563 A1
(43) Pub. Date: Sep. 27, 2007

(54) INSERTION PORTION OF AN ENDOSCOPE

(75) Inventor: Takayuki OGINO, Saitama (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/688,367

(22) Filed: Mar. 20, 2007

(30) Foreign Application Priority Data
Mar. 22, 2006 (JP) 2006-077914

(51) Int. Cl. A61B 1/00; A61B 1/04

(52) U.S. Cl. 600/130; 600/142

(57) ABSTRACT

An insertion portion of an endoscope includes a bendable flexible tubular framework composed of a plurality of short-cylindrical joint rings which are coupled in series via rotatable connecting shafts, and a plurality of flexible internal elements which are inserted into the flexible tubular framework and arranged to extend straightly in an axial direction of the flexible tubular framework upon distal ends of the flexible internal elements being fixed to a distal end of the insertion portion. Inner end surfaces of each of the connecting shafts are positioned between two adjacent flexible internal elements of the flexible internal elements around a perimeter of the plurality of flexible internal elements.

### BACKGROUND OF THE INVENTION

1. Field of the Invention

The present invention relates to an insertion portion of an endoscope.

2. Description of the Related Art

An insertion portion of an endoscope which is introduced into a body is generally constructed of a metal helical tube, a mesh tube and a flexible jacket, wherein the metal helical tube is sheathed with the mesh tube and further sheathed with the flexible jacket. However, in the case where the endoscope is sterilized by a process of steaming under pressure (autoclaving), the metal helical tube may shrink. Accordingly, to achieve durability against autoclaving, the helical tube may be replaced by a flexible tubular framework consisting of a plurality of short-cylindrical joint rings which are coupled in series via connecting shafts to be freely rotatable (as shown in, e.g., Japanese unexamined patent publication H09-24020).

It is desirable that various internal elements which are inserted into the inserted portion of the endoscope to be installed therein be inserted straightly into the inserted portion from the distal fixing ends of the various elements because the various internal elements may be twisted and tangled to thereby be easily damaged if the relative positions of the various internal elements are changed during the insertion of the various internal elements into the inserted portion of the endoscope.

However, in the case of the insertion portion of the endoscope using the aforementioned flexible tubular framework, which consists of a plurality of short-cylindrical joint rings which are coupled in series via connecting shafts to be freely rotatable, instead of using a helical tube, the internal structure of the insertion portion is such that the inner end surfaces (radially inner end surfaces) of connecting shafts 91 project radially inwards from the inner peripheral surface of the associated joint ring 92 as shown in FIG. 6.

Therefore, among the various internal elements inserted into the aforementioned flexible tubular framework (91 and 92), internal elements 100 which are arranged at positions facing the inner end surfaces of the connecting shafts 91 may interfere with the connecting shafts 91, respectively, and forcibly moved thereby radially inwards thereby if the internal elements 100 are inserted straightly into the insertion portion of the endoscope to pass therethrough from the distal fixing ends of the internal elements 100.

Consequently, all the internal elements positioned in the flexible tubular framework (91 and 92) are in a tightly-packed state as a whole, so that there is a possibility of the internal elements being damaged (e.g., there is a possibility of a bundle of optical fibers, a tube and a signal cable being damaged by a breakage, buckling and breaking, respectively) when, e.g., the flexible tubular framework is shaped into a loop.

### SUMMARY OF THE INVENTION

The present invention provides an insertion portion of an endoscope which uses a flexible tubular framework instead of a helical tube, wherein the flexible tubular framework is composed of a plurality of joint rings which are coupled in series via connecting shafts to be freely rotatable, and wherein the internal elements of the insertion portion are smoothly inserted into the insertion portion to be installed therein, thus being unsusceptible to damage, and superior in durability.

According to an aspect of the present invention, an insertion portion of an endoscope is provided, including a bendable flexible tubular framework composed of a plurality of short-cylindrical joint rings which are coupled in series via rotatable connecting shafts, and a plurality of flexible internal elements which are inserted into the flexible tubular framework and arranged to extend straightly in an axial direction of the flexible tubular framework upon distal ends of the flexible internal elements being fixed to a distal end of the insertion portion. Inner end surfaces of each of the connecting shafts are positioned between two adjacent flexible internal elements of the flexible internal elements around a perimeter of the plurality of flexible internal elements.

It is desirable for the connecting shafts to include a first pair of radially-opposed connecting shafts provided on each of the plurality of joint rings; and a second pair of radially-opposed connecting shafts provided on the each of the plurality of joint rings, a common axis of the first pair of radially-opposed connecting shafts extending at a different angle relative to a common axis of the second pair of radially-opposed connecting shafts as viewed in an axial direction of the each of the plurality of joint rings. The first pair of radially-opposed connecting shafts, provided on the plurality of joint rings, are aligned in a direction orthogonal to an axial direction of the tubular frame work, and the second pair of radially-opposed connecting shafts, provided on the plurality of joint rings, are aligned in the orthogonal direction.

It is desirable for the plurality of flexible internal elements to be fixed to an end body of the insertion portion at the distal fixing ends, respectively, to extend straightly from the distal fixing ends wherein relative positions of the plurality of flexible internal elements in the flexible tubular framework remain unchanged.

It is desirable for each of the plurality of joint rings to be coupled at one end thereof to one adjacent joint ring of the plurality of joint rings via two connecting shafts of the connecting shafts at 180-degree symmetrical positions, respectively, and is coupled at another end thereof to another adjacent joint ring of the plurality of joint rings via another two connecting shafts of the connecting shafts at 180-degree symmetrical positions, respectively, with a predetermined degree of phase shift with respect to the one adjacent joint ring It is desirable for the predetermined degree of phase shift to be 90-degree phase shift It is desirable for the plurality of flexible internal elements to include at least one optical fiber bundle.

It is desirable for the plurality of flexible internal elements to include at least one flexible tube for fluid transmission.

It is desirable for the plurality of flexible internal elements to include at least one electric signal cable.

It is desirable for each of the plurality of joint rings to include a first pair of tongues positioned at one end of the joint ring symmetrically at 180-degree positions, respectively, and for a second pair of tongues positioned at another end of the joint ring symmetrically at 180-degree positions, respectively, at the predetermined degree of phase shift with respect to the one adjacent joint ring. Two through-holes through which the two connecting shafts pass are formed in the first pair of tongues, respectively, and another two through-holes through which the another two connecting shafts pass are formed in the second pair of tongues, respectively.

It is desirable for an insertion portion of an endoscope to be provided, which includes a bendable flexible tubular framework composed of a plurality of short-cylindrical joint rings which are coupled in series via connecting shafts, a plurality of flexible internal elements being inserted into the flexible tubular framework and installed therein in a bundle configuration. The plurality of flexible internal elements are arranged in the flexible tubular framework to extend straightly parallel to one another so that an axis of each of the connecting shaft that extends in a radial direction of the insertion portion of the endoscope is positioned in between axes of two adjacent flexible internal elements of the plurality of flexible internal elements that extend in an axial direction of the insertion portion of the endoscope.

According to the present invention, since a plurality of flexible internal elements are inserted straightly into the flexible tubular framework to pass therethrough straightly from the distal fixing ends of the plurality of internal elements and arranged so that the inner end surfaces of the connecting shafts are positioned with clearances created among adjacent internal elements around the perimeter of the bundle of internal elements, the internal elements of the insertion portion are smoothly inserted into the insertion portion to be installed therein, thus being unsusceptible to damage, and superior in durability in the insertion portion of an endoscope using a flexible tubular framework composed of a plurality of joint rings which are coupled in series via connecting shafts to be freely rotatable instead of using a helical tube.

The present disclosure relates to subject matter contained in Japanese Patent Application No. 2006-77914 (filed on Mar. 22, 2006) which is expressly incorporated herein by reference in its entirety.

### BRIEF DESCRIPTION OF THE DRAWINGS

The present invention will be discussed below in detail with reference to the accompanying drawings, in which:

FIG. 1 is a cross sectional view of an insertion portion of an embodiment of an endoscope according to the present invention, taken along a plane orthogonal to the axis of the insertion portion flexible tube (taken along I-I line shown in FIG. 4);

FIG. 2 is a front elevational view of the end surface of the end body provided at the front end of the insertion portion of the endoscope according to the present invention;

FIG. 3 is a side view of the endoscope according to the present invention, showing the overall structure thereof;

FIG. 4 is a side elevational view, partly in cross section, of the flexible tubular framework of the insertion portion of the endoscope according to the present invention;

FIG. 5 is a perspective view of a joint ring of the flexible tubular framework of the insertion portion of the endoscope according to the present invention; and

FIG. 6 is a cross sectional view of an insertion portion of a conventional endoscope, taken along a plane orthogonal to the axis of the flexible tubular framework of the insertion portion.

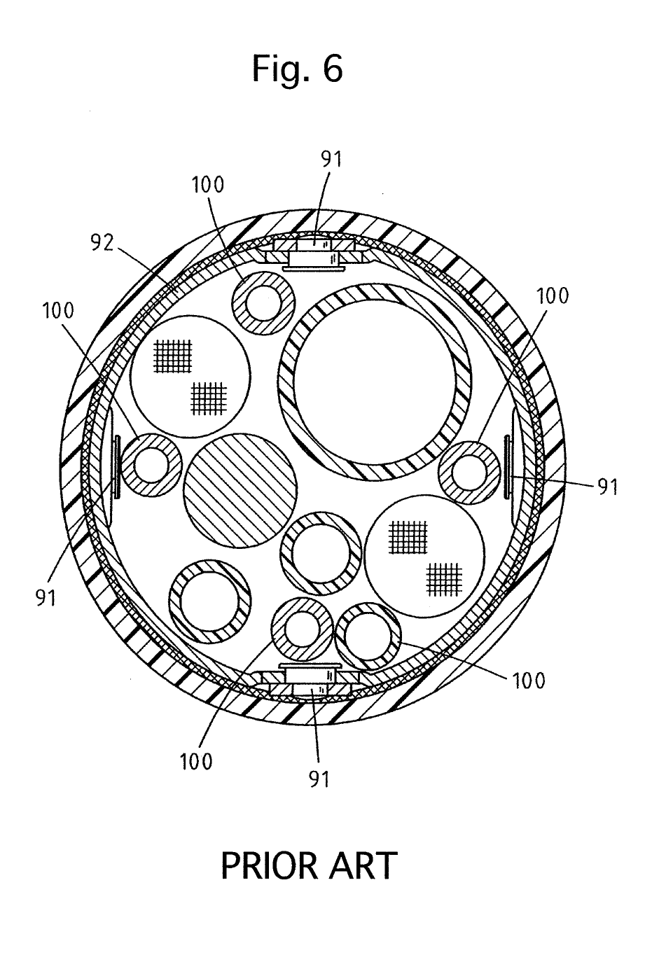

### PREFERRED EMBODIMENT OF THE PRESENT INVENTION

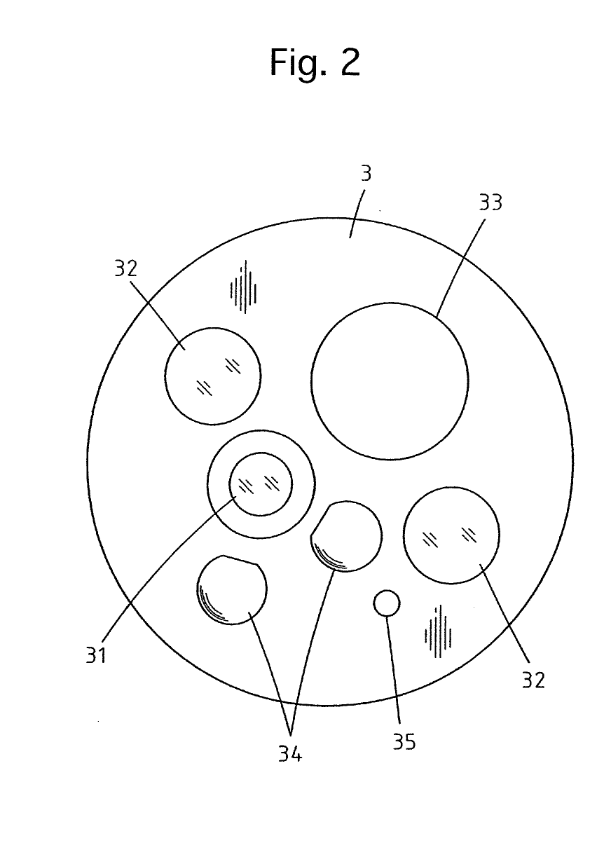

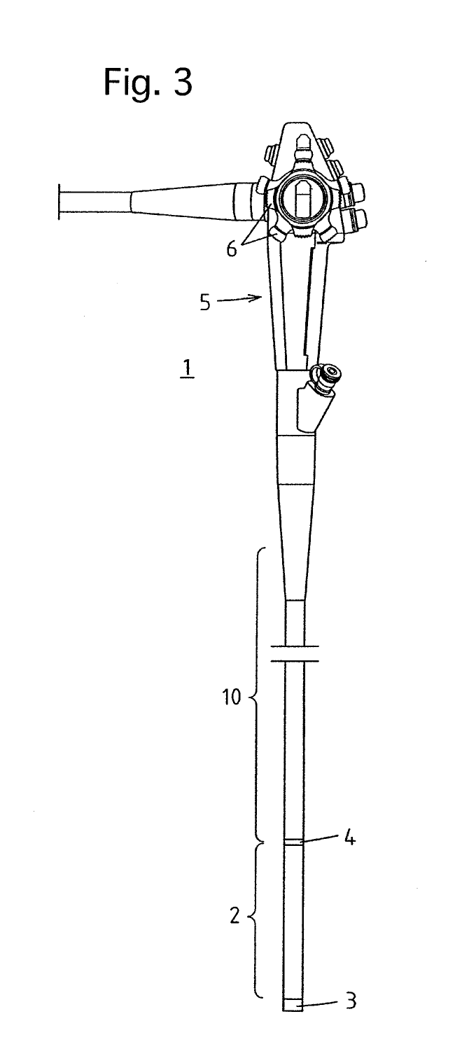

FIG. 3 shows the overall structure of the endoscope 1 according to the present invention. The endoscope 1 is provided with a control body 5 and an insertion portion connected to the control body 5. The distal end portion of the insertion portion is formed as a steerable bendable portion 2. The insertion portion of the endoscope 1 is provided with a flexible tubular portion 10, and the steerable bendable portion 2 is joined to the front end of the flexible tubular portion 10. The insertion portion of the endoscope 1 is further provided at the end of the steerable bendable portion 2 with an end body 3 in which an objective window 31, two illumination windows 32, a treatment tool exit mouth 33, two air/water supply nozzles 34 and a sub-water supply nozzle 35 are provided (see FIG. 2). The flexible tubular portion 10 and the steerable bendable portion 2 are connected to each other via a connecting ring 4. The steerable bendable portion 2 can be steered to bend freely (right, left, upward and downward) by controlling steering knobs 6 provided on the control body 5.

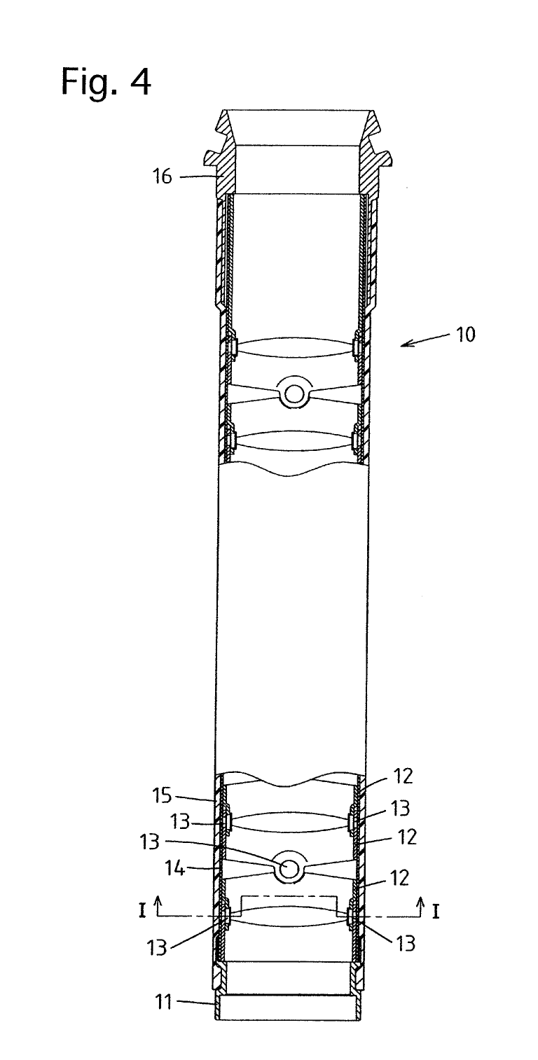

FIG. 4 shows the flexible tubular portion 10. The flexible tubular portion 10 in this embodiment of the endoscope is provided with a freely-bendable flexible tubular framework composed of a plurality (e.g., several dozen to several hundreds) of short-cylindrical joint rings 12 which are coupled in series via connecting shafts 13 to be freely rotatable. The axis of each shaft 13 is orientated to pass through the axis of the associated joint ring 12. For instance, the connecting shafts 13 can be formed as rivets. The outer periphery of the freely-bendable flexible tubular framework is sheathed with a braided sheath (mesh tube) 14 which is formed by braiding fine metal strands, and the outer periphery of the braided sheath 14 is sheathed with a flexible jacket 15.

The flexible tubular portion 10 is provided at a distal end (bottom end as viewed in FIGS. 3 and 4) thereof with a distal-end mouth ring 11 which is formed to be connected to the steerable bendable portion 2 via the connecting ring 4. The flexible tubular portion 10 is provided at a proximal end (top end as viewed in FIGS. 3 and 4) thereof with a proximal-end mouth ring 16 which is formed to be connected to the control body 5. Although various internal elements which will be discussed later are inserted into the flexible tubular portion 10 to be installed therein, such internal elements are not shown in FIG. 4 for the purpose of clarity.

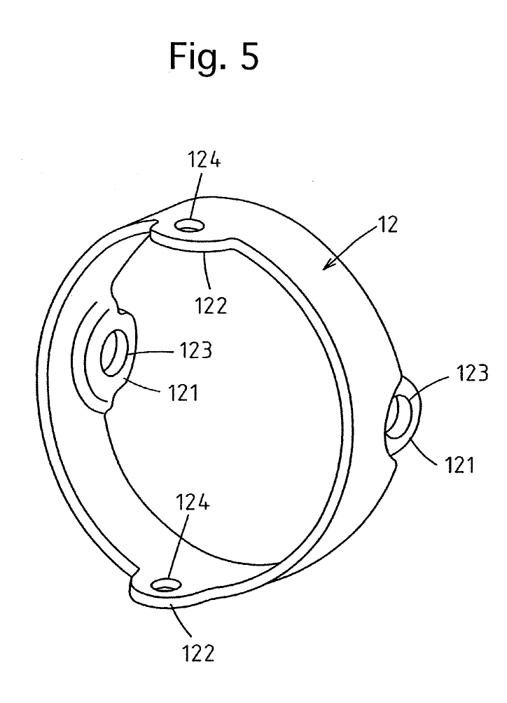

For instance, each of the plurality of short-cylindrical joint rings 12 is formed as the shape as shown in FIG. 5. Namely, the joint ring 12 is provided at one end thereof with a pair of tongues 121 which are positioned symmetrically at 180-degree positions, respectively, and is further provided at the other end thereof with a pair of tongues 122 which are positioned symmetrically at 180-degree positions, respectively, at a 90-degree phase shift with respect to the pair of tongues 121 about the axis of the joint ring 12.

The pair of tongues 121 are formed in a manner such that the radially outer surfaces thereof are each recessed radially inwards by the amount of thickness of each of the pair of tongues 122 so that the pair of tongues 122 of one joint ring 12 are overlaid on the pair of tongues 121 of an adjacent joint ring 12 with the radially outer surfaces of the pair of tongues 122 being substantially flush with the outer peripheral surface of the adjacent joint ring 12 when the plurality of short-cylindrical joint rings 12 are arranged in series so that the axes thereof are aligned. A through-hole 123 through which one connecting shaft 13 passes is made in each tongue 121 and a through-hole 124 through which one connecting shaft 13 passes is made in each tongue 122.

Therefore, each joint ring 12 is coupled at one end thereof to one adjacent joint ring 12 via two connecting shafts (a first pair of radially-opposed connecting shafts) 13 at 180-degree symmetrical positions, respectively, and is further coupled at the other end thereof to another adjacent joint ring 12 via another two connecting shafts (a second pair of radially-opposed connecting shafts) 13 at 180-degree symmetrical positions, respectively, with a 90-degree phase shift with respect to the aforementioned adjacent joint ring 12 Accordingly, the first pair of radially-opposed connecting shafts 13 are aligned and the common axis thereof that passes through the axis of the associated joint ring 12. Likewise, the second pair of radially-opposed connecting shafts 13 are aligned and the common axis thereof that passes through the axis of the associated joint ring 12. The first pairs of radially-opposed connecting shafts 13 of the plurality of joint rings 12 connected in series are aligned in the axial direction of the freely-bendable flexible tubular framework, respectively. Likewise, the second pairs of radially-opposed connecting shafts 13 of the plurality of joint rings 12 connected in series are aligned in the axial direction of the freely-bendable flexible tubular framework, respectively. However, it is possible that the phase angle between two joint rings 12 coupled to each joint ring 12 at the opposite ends thereof, respectively, be any phase angle other than 90-degree angle (e.g., 45-degree angle or 60-degree angle).

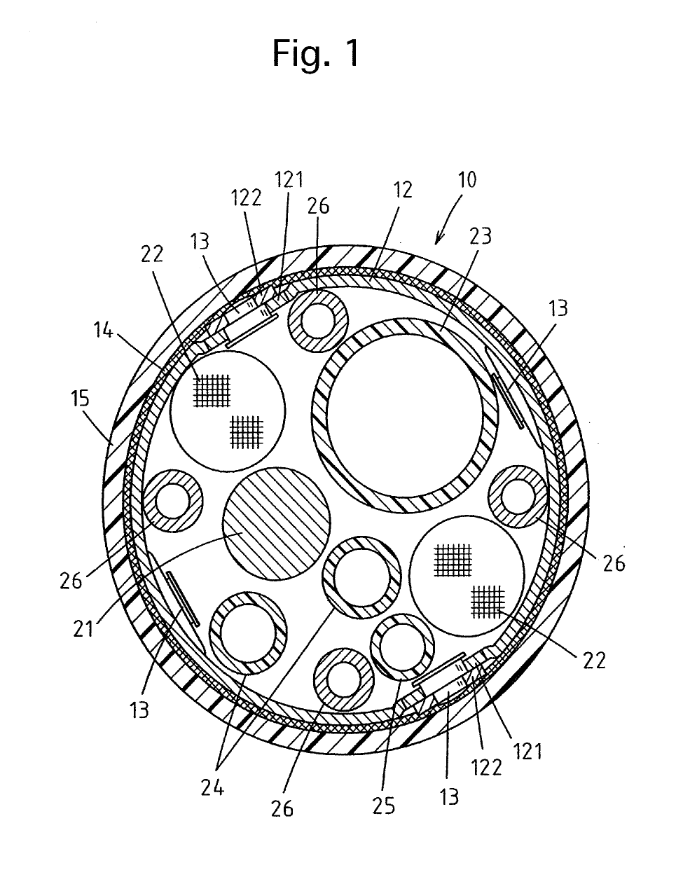

FIG. 1 is a cross sectional view of the flexible tubular portion 10 in which various internal elements (flexible internal elements) have been inserted and installed, taken along I-I line shown in FIG. 4. The various internal elements include an electric signal cable 21 for transmitting signals such as an imaging signal, two optical fiber bundles 22 for lighting, a treatment tool insertion channel 23 into which a treatment tool (not shown) is inserted, two air/water supply tubes 24 each of which is capable of serving as an air supply tube or a water supply tube for blowing air or spraying water to the surface of the objective window 31, a sub-water supply tube 25 for spraying water toward the front of the end body 3, and four guide coils 26 through which four control wires (not shown) which are moved to manipulate the steerable bendable portion 2 by manually operating the steering knobs 6. All these internal elements (21 through 26) have high flexibility.

FIG. 2 shows the end surface of the end body 3. As shown in FIG. 2, the end body 3 is provided with the objective window 31 for capturing object images, the two illumination windows 32 for emitting illumination light, the treatment tool exit mouth 33 from which the aforementioned treatment tool (not shown) that is inserted into the treatment tool insertion channel 23 can jut out, the two air/water supply nozzles 34 which are open toward the surface of the objective window 31, and the sub-water supply nozzle 35 which is open straight toward the front of the end body 3.

The end body 3 is provided therein behind the objective window 31 with an objective lens and a solid-state image pickup device (both not shown), and the front end of the electric signal cable 21 is connected to the end body 3 and fixed thereto at a position behind the solid-state image pickup device. The exit ends (not shown) of the two optical fiber bundles 22 are connected to the end body 3 and fixed thereto at two positions behind the two illumination windows 32, respectively.

The exit end (not shown) of the treatment tool insertion channel 23 is positioned in the inner part the treatment tool exit mouth 33 to be communicatively connected thereto and fixed to the end body 3. The exit ends (not shown) of the two air/water supply tubes 24 are positioned in the inner parts of the two air/water supply nozzles 34 to be communicatively connected thereto, respectively, and fixed to the end body 3. The exit end (not shown) of the sub-water supply tube 25 is positioned in the inner part of the sub-water supply nozzle 35 to be communicatively connected thereto, and fixed to the end body 3.

The electric signal cable 21, the two optical fiber bundles 22, the treatment tool insertion channel 23, the two air/water supply tubes 24 and the sub-water supply tube 25, which are fixed to the end body 3 in the above-described manner, are inserted straightly into the flexible tubular portion 10 to run therethrough in a direction parallel to the axes of the end body 3, the steerable bendable portion 2 and the flexible tubular portion 10.

Each of the four guide coils 26 fixed to the inner peripheral surface of the connecting ring 4 (these fixing portions are not shown) is inserted straightly into the flexible tubular portion 10 to run therethrough in a direction parallel to the axis of the flexible tubular portion 10.

Therefore, all the internal elements (21 through 26) are each inserted into the flexible tubular framework (12 and 13) to pass therethrough straightly from the distal fixing ends of the internal elements (21 through 26). Additionally, as shown in FIG. 1, the bundle of internal elements (21 through 26) are positioned and arranged so that the inner end surfaces (radially inner end surfaces) of all the connecting shafts 13 are positioned between adjacent internal elements around the perimeter of the bundle of internal elements (21 through 26). In other words, the internal elements (21 through 26) are arranged in the flexible tubular framework (12 and 13) to extend straightly parallel to one another so that an axis of each of the connecting shaft 13 that extends in a radial direction of the insertion portion of the endoscope 1 is positioned in between axes of two adjacent internal elements (21 through 26). For instance, regarding the lower left connecting shaft 13 as viewed in FIG. 1, the radially inner end surface of this connecting shaft 13 is positioned in a space defined between one of the two air/water supply tubes 24 and an adjacent one of the four guide coils 26.

In other words, each of the internal elements (21 through 26) is set so that the inner end surface of each connecting shaft 13 is positioned between adjacent internal elements of the internal elements (21 through 26), and each of the internal elements (21 through 26) is fixed to the end body 3 at the distal fixing end to extend straightly from the distal fixing end.

Consequently, even in the case of the insertion portion of the endoscope using the aforementioned flexible tubular framework, that is composed of the plurality of joint rings 12 which are coupled in series via the connecting shafts 13 to be freely rotatable, instead of using a helical tube, the bundle of internal elements (21 through 26) are not tightly packed as a whole in the flexible tubular portion 10, and accordingly, the effect of preventing the optical fiber bundle 22 from being damaged by a breakage, the effect of preventing the treatment tool insertion channel 23, the two air/water supply tubes 24 and the sub-water supply tube 25 from buckling, and the effect of preventing the electric signal cable 21 from breaking even when, e.g., the flexible tubular framework is shaped into a loop can be obtained.

Obvious changes may be made in the specific embodiment of the present invention described herein, such modifications being within the spirit and scope of the invention claimed. It is indicated that all matter contained herein is illustrative and does not limit the scope of the present invention.

What is claimed is:

1. An insertion portion of an endoscope comprising:
a bendable flexible tubular framework composed of a plurality of short-cylindrical joint rings which are coupled in series via rotatable connecting shafts, and
a plurality of flexible internal elements which are inserted into said flexible tubular framework and arranged to extend straightly in an axial direction of said flexible tubular framework upon distal ends of said flexible internal elements being fixed to a distal end of said insertion portion,
wherein inner end surfaces of each of said connecting shafts are positioned between two adjacent flexible internal elements of said flexible internal elements around a perimeter of said plurality of flexible internal elements.

2. The insertion portion of the endoscope according to claim 1, wherein said connecting shafts comprise:
a first pair of radially-opposed connecting shafts provided on each of said plurality of joint rings; and
a second pair of radially-opposed connecting shafts provided on said each of said plurality of joint rings, a common axis of said first pair of radially-opposed connecting shafts extending at a different angle relative to a common axis of said second pair of radially-opposed connecting shafts as viewed in an axial direction of said each of said plurality of joint rings,
wherein said first pair of radially-opposed connecting shafts, provided on said plurality of joint rings, are aligned in a direction orthogonal to an axial direction of said tubular frame work, and
wherein said second pairs of radially-opposed connecting shafts, provided on said plurality of joint rings, are aligned in said orthogonal direction.

3. The insertion portion of the endoscope according to claim 1, wherein said plurality of flexible internal elements are fixed to an end body of said insertion portion at said distal fixing ends, respectively, to extend straightly from said distal fixing ends wherein relative positions of said plurality of flexible internal elements in said flexible tubular framework remain unchanged.

4. The insertion portion of the endoscope according to claim 1, wherein each of said plurality of joint rings is coupled at one end thereof to one adjacent joint ring of said plurality of joint rings via two connecting shafts of said connecting shafts at 180-degree symmetrical positions, respectively, and is coupled at another end thereof to another adjacent joint ring of said plurality of joint rings via another two connecting shafts of said connecting shafts at 180-degree symmetrical positions, respectively, with a predetermined degree of phase shift with respect to said one adjacent joint ring

5. The insertion portion of the endoscope according to claim 4, wherein said predetermined degree of phase shift is 90-degree phase shift

6. The insertion portion of the endoscope according to claim 1, wherein said plurality of flexible internal elements comprise at least one optical fiber bundle.

7. The insertion portion of the endoscope according to claim 6, wherein said plurality of flexible internal elements comprise at least one flexible tube for fluid transmission.

8. The insertion portion of the endoscope according to claim 6, wherein said plurality of flexible internal elements comprise at least one electric signal cable.

9. The insertion portion of the endoscope according to claim 4, wherein each of said plurality of joint rings comprises:
a first pair of tongues positioned at one end of said joint ring symmetrically at 180-degree positions, respectively, and
a second pair of tongues positioned at another end of said joint ring symmetrically at 180-degree positions, respectively, at said predetermined degree of phase shift with respect to said one adjacent joint ring,
wherein two through-holes through which said two connecting shafts pass are formed in said first pair of tongues, respectively, and another two through-holes through which said another two connecting shafts pass are formed in said second pair of tongues, respectively.

10. An insertion portion of an endoscope which includes a bendable flexible tubular framework composed of a plurality of short-cylindrical joint rings which are coupled in series via connecting shafts, and a plurality of flexible internal elements being inserted into said flexible tubular framework and installed therein in a bundle configuration,
wherein said plurality of flexible internal elements are arranged in said flexible tubular framework to extend straightly parallel to one another so that an axis of each of said connecting shaft that extends in a radial direction of said insertion portion of said endoscope is positioned in between axes of two adjacent flexible internal elements of said plurality of flexible internal elements that extend in an axial direction of said insertion portion of said endoscope.
